(19) United States
(12) Patent Application Publication
(10) Pub. No.: US 2008/0188877 A1
(43) Pub. Date: Aug. 7, 2008

(54) Instruments For Removing an Object From the Eye

(76) Inventor: Dyson W. Hickingbotham, Stouchsburg, PA (US)

Correspondence Address: ALCON, IP LEGAL, TB4-8, 6201 SOUTH FREEWAY, FORT WORTH, TX, 76134, US

(21) Appl. No.: 11/671,019

(22) Filed: Feb. 5, 2007

(51) Int. Cl. A61F 9/007

(52) U.S. Cl. 606/162; 606/207; 606/170

(57) ABSTRACT

The present invention provides an instrument set for removing an object from the eye including a forceps having first and second jaws mounted for relative movement toward and away from one another between a first position in which the backs of the jaws are relatively nearer to one another and a second position in which the backs of the jaws are relatively further from one another. At least one of the jaws defines a cutter on its back able to cut an exit wound upon withdrawal of the jaws from the eye. The forceps may optionally include a conforming portion softer than the jaw body. The instrument set may optionally include elongated magnetic probe. The instrument set may optionally be provided in a single procedure pack that encloses the forceps and probe in a closed sterile environment. A method for removing an object from an eye includes grasping the object with the forceps and withdrawing the forceps and object from the eye while simultaneously cutting an exit wound with a cutter mounted to the back of at least one of the jaws. The method may optionally include transferring the object to the forceps with the magnetic probe.

### FIELD OF THE INVENTION

The invention relates to instruments for performing surgery on the eye. In particular, the invention relates to instruments for surgically removing an object or foreign body from the eye.

### BACKGROUND OF THE INVENTION

Removal of foreign bodies from the eye is a common surgical procedure. For example, intraocular foreign bodies are commonly introduced into the eye by industrial, construction, automobile, firearms, and other types of accidents. Removal of the foreign body is typically necessary to prevent further injury and facilitate repair of the eye. Foreign bodies may be magnetic or non-magnetic. Small magnetic foreign bodies are typically extracted using a magnetic probe. The probe may include a retractable magnet to permit selective attraction and release of the magnetic foreign body. Retractable magnetic probes tend to be relatively expensive devices to produce and are most suitable for durable or multiple use instruments that are cleaned and reused a number of times. Durable or multiple use instruments require proper cleaning and maintenance so that they are ready for use when needed.

Non-magnetic foreign bodies require gripping such as between opposing jaws. Furthermore, the circumstances of the foreign body injury, or surgeon preference, may cause the surgeon to opt for a gripping instrument even for magnetic foreign bodies. Large foreign bodies that must be gripped can be difficult to extract. In particular, sharp slippery foreign bodies, such as glass particles, can be difficult to grip and extract. In addition to the initial difficulty of gripping the foreign body, slippage of the foreign body can occur and result in further injury to the eye. One solution to gripping difficult foreign bodies has been the use of forceps with diamond coated jaws. The hard, irregular surface of the diamond coating bites into and grips the hard, slippery exterior of the foreign body. While effective, such diamond coated forceps tend to be relatively expensive to produce and, as with retractable magnetic probes, are most suitable for durable or multiple use instruments.

In a typical operation, the foreign body entry wound is repaired. A sclerotomy is made in the globe of the eye to receive fluid inflow so that eye pressure can be managed. Additional sclerotomies are made to receive surgical instruments. An initial instrument entry sclerotomy is preferably just large enough to admit the instrument. Vitrectomy, posterior vitreous detachment, peeling of membranes, and other procedures known in the art are carried out to prepare for removal of the foreign body and encourage eye healing. After gripping the foreign body, the surgeon estimates the exit wound size necessary to extract the gripping jaws and foreign body. Accuracy in estimating the exit wound size is important to avoid making too large of an exit wound and creating unnecessary tissue trauma. Likewise, it is important to avoid making too small of an exit wound which could result in tearing of the sclera or cause the foreign body to be dislodged from the extraction instrument causing additional eye damage. The surgeon enlarges the exit wound to the estimated size and extracts the foreign body. The exit wound is repaired and additional procedures may be performed such as intraocular lens implantation to complete the ocular repair. Lens implantation and other interventions may also be performed in secondary procedures after the eye has had an opportunity to heal from the initial extraction.

Other objects that may require removal from the eye include objects introduced during an ocular surgical procedure and resected tissue. Throughout this specification, the term “foreign body” is used to refer to objects being removed from the eye and includes all kinds of objects. Such objects include those accidentally introduced into the eye, those therapeutically introduced into the eye, tissue resected from the eye, and all other objects. The terms “proximal” and “distal” are used to describe positions relative to the end of an instrument that engages the eye. “Proximal” is used to refer to positions nearer the working end and “distal” is used to refer to positions further from the working end.

### SUMMARY OF THE INVENTION

The present invention provides an instrument for removing an object from the eye.

In one aspect of the invention, a forceps includes first and second jaws mounted for relative movement toward and away from one another between a first position in which the backs of the jaws are relatively nearer to one another and a second position in which the backs of the jaws are relatively further from one another. At least one of the jaws defines a cutter on its back able to cut an exit wound upon withdrawal of the jaws from the eye.

In another aspect of the invention, the forceps includes at least one jaw having a jaw face including a conforming portion softer than the jaw body.

In another aspect of the invention, an instrument set includes the forceps and an elongated probe. The probe includes a magnet translatable relative to a sleeve from a first position in which at least a portion of the magnet projects beyond the sleeve and a second position in which the magnet is retracted within the sleeve.

In another aspect of the invention, the forceps and the probe are sterile and packaged together in a single procedure pack that encloses the forceps and probe in a closed sterile environment.

In another aspect of the invention, a method for removing an object from an eye includes grasping the object with the forceps and withdrawing the forceps and object from the eye while simultaneously cutting an exit wound with a cutter mounted to the back of at least one of the jaws.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a perspective view of set, or procedure pack, of ocular instruments according to the present invention including a forceps and a retractable magnetic probe;

FIG. 2 is a cross-sectional view of the forceps of FIG. 1;

FIG. 3 is a an enlarged detail view of a portion of the forceps of FIG. 1;

FIG. 4 is a cross-sectional view of the retractable magnetic probe of FIG. 1; and

FIGS. 5-8 are cut-away views of an eye illustrating a surgical procedure performed using the ocular instruments of FIG. 1.

### DETAILED DESCRIPTION OF THE EMBODIMENTS

Embodiments of the ocular foreign body removal instruments according to the present invention may include a foreign body forceps and/or a retractable magnetic probe.

The forceps may include two or more opposing jaws for gripping an object. The jaws may be mounted for relative movement away from and toward one another to permit the jaws being opened to receive the object between them and to permit the jaws being closed to grip the object. The jaws may be mounted for relative pivoting and/or translating movement. The jaws may be mounted in a scissor-like arrangement for simple angular pivoting relative to one another, in a four-bar parallel linkage arrangement for parallel relative translation of the jaws, in a relative sliding relationship, in a sprung cantilevered arrangement, and/or in any other suitable arrangement allowing the jaws to move toward and away from one another. For example, the jaws may be defined by the ends of a resilient “Y”-shaped member that flexes as the jaws are moved toward and away from one another. A sleeve may be mounted over the “Y”-shaped member to actuate the jaws such that relative movement of the sleeve and jaws toward one another compresses the ends of the “Y”-shaped member toward one another to close the jaws and relative movement of the sleeve and jaws away from one another allows the ends of the “Y”-shaped member to spring away from one another to open the jaws. The sleeve and “Y”-shaped member may be mounted to a handle and one of the sleeve and “Y”-shaped member may be fixed relative to the handle while the other is movable relative to the handle. For example, the “Y”-shaped member may be fixed relative to the handle and the sleeve may be movable relative to the handle such that the jaws are operable without changing the position of the jaws relative to the handle. While this arrangement facilitates fine control of the placement of the jaws intraoperatively, the sleeve may be fixed and the jaws may be movable. The jaws may include inwardly facing jaw faces for gripping the object and outwardly facing jaw backs.

One or more of the jaw backs may include a cutter that cuts an exit wound sized to receive the jaws upon extraction of the forceps from the eye. A cutter on one jaw back cuts an exit wound in one direction while a cutter on multiple jaw backs cuts an exit wound in multiple directions. For example, opposing cutters may each include a cutter on the jaw back to create an exit wound in opposing directions. Cutters may be hinged so that a blade appears when an object is gripped. The cutters may include burrs, blades, and/or other cutters. Elongated blade cutters may extend for a distance that is shorter than the overall length of the jaw backs, a distance equal to the length of the jaw backs, and/or a distance that is greater than the length of the jaw backs. The cutters may be straight or tapered. Tapered cutters may be tapered over a portion of their length or along their entire length. For example, the cutters may include elongated thin blades projecting outwardly from and extending along the length of the jaw backs from near the open end of the jaws toward the handle with a tapering end portion toward the handles to initiate the cut.

The jaw faces may be flat, curved, smooth, textured, and/or otherwise shaped. For example, textured jaw faces may include, etched surfaces, embedded particles, forged teeth, cut teeth, and/or other suitable texturing. The jaw faces may include a relatively soft surface that deforms to grip a relatively hard object. For example the jaw faces may include a relatively soft polymer, metal, latex, and/or other material. The jaw faces may be softer than the rest of the jaw body such that the jaw body is relatively strong while the jaw face is elastically and/or plastically deformable to grip an object. A polymer jaw face may include polyolefins, polyesters, polyimides, polyamides, polyacrylates, poly(ketones), fluropolymers, synthetic rubbers, polysiloxanes, and/or any other suitable polymers. For example the jaw faces may be made of silicone. For example the jaw faces be relatively flat with outwardly projecting teeth formed on them. Soft jaw faces may be welded, bonded, molded, mechanically fastened, and/or otherwise attached to the jaws. For example, polymer jaw faces may be molded to the jaws. The forceps may be made primarily of inexpensive materials and moldings to reduce the instrument cost to the point of disposability.

The retractable magnetic probe may include an elongated shaft with a magnet mounted at its tip for attracting magnetic objects. A sleeve may be mounted over shaft in relative sliding relationship to permit selective attraction and release of magnetic objects. With the shaft and sleeve positioned to expose the magnet, magnetic objects are attracted relatively more strongly to the magnet and may contact the magnet for maximum holding power. With the shaft and sleeve positioned to conceal the magnet, magnetic objects are attracted relatively less strongly to the magnet and the sleeve may act to dislodge objects from the magnet. The sleeve and shaft may be mounted to a handle and one of the sleeve and shaft may be fixed relative to the handle while the other is movable relative to the handle between a position in which the magnet is exposed at the tip of the shaft and a position in which the magnet is covered to permit selective attraction and release of a magnetic object. For example, the sleeve may be fixed relative to the handle and the shaft may be movable relative to the handle such that moving the shaft into the sleeve tends to dislodge objects from the magnet as the magnet moves into the end of the sleeve. Alternatively, the shaft may be fixed and the sleeve may be movable. The retractable magnetic probe may be made primarily of inexpensive materials and moldings to reduce the instrument cost to the point of disposability.

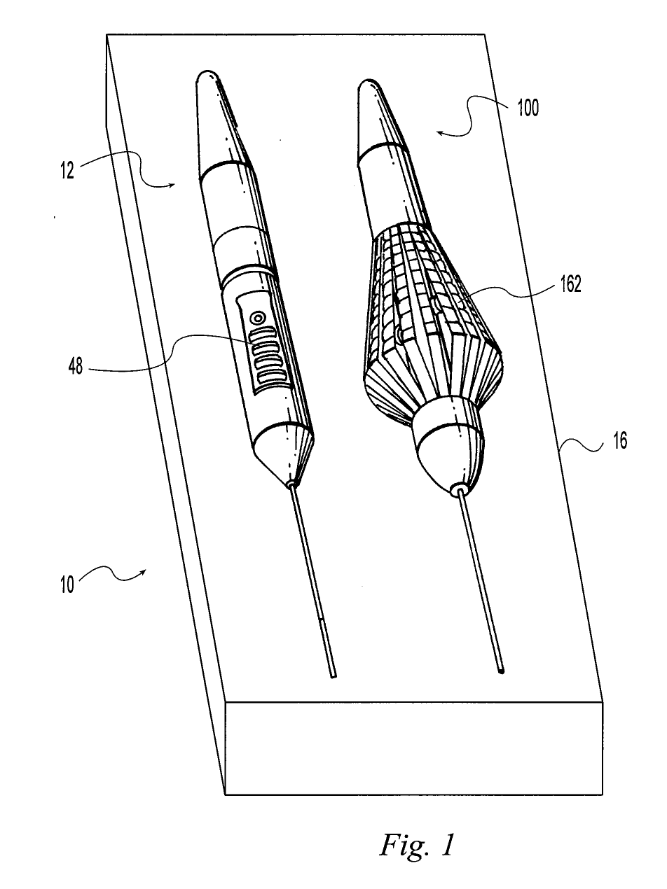

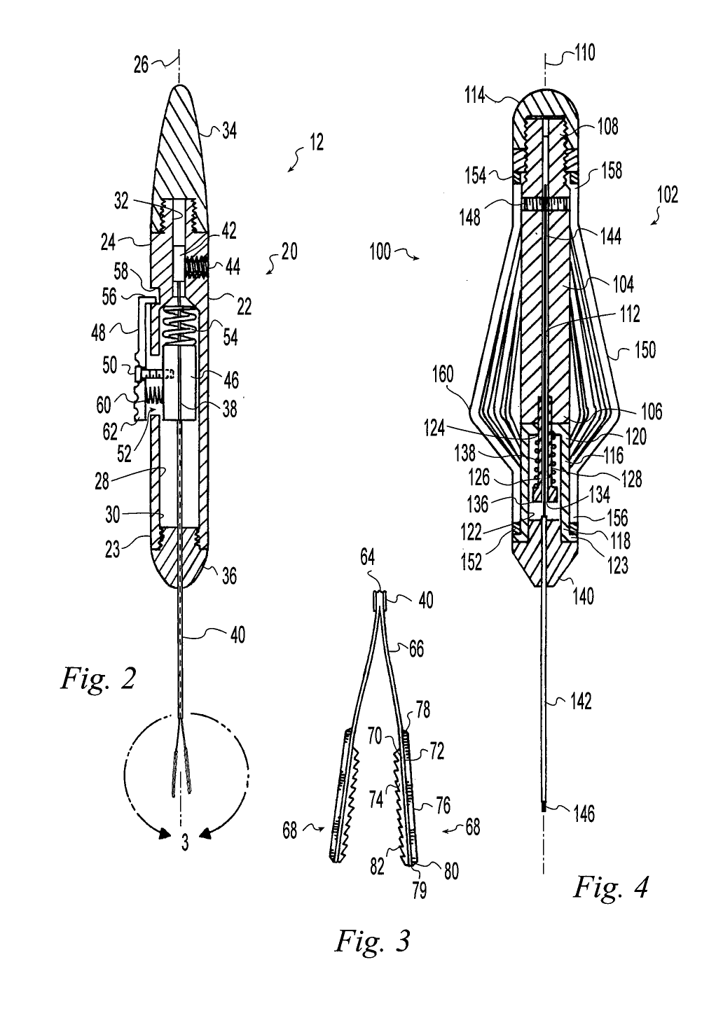

FIGS. 1-4 depict an illustrative embodiment of a set 10 of surgical instruments for removing foreign bodies from the eye. The instruments include a foreign body forceps 12 and a retractable magnetic probe 100 packaged in a procedure pack 16. Preferably the instruments are provided as a single use sterile pack 16 that insures the instruments are clean and ready for immediate use. This is particularly helpful due to the small size of the instrument components and the relative complexity of cleaning and maintaining them.

The forceps 12 includes a handle 20 including a hollow body tube 22 having a proximal end 23, a distal end 24, and an axis 26 extending therebetween. The body tube 22 includes a stepped axial through bore 28 with a larger diameter proximal end 30 and a smaller diameter distal end 32. An end cap 34 threads onto the distal end 24 of the body tube 22 to close the axial through bore 28 distally. A cannulated nose piece 36 threads onto the proximal end 23 of the body tube 22 to reduce the axial through bore 28 proximally. A jaw shaft 38 and sleeve 40 are coaxially mounted within the body tube 22 and extend through the cannulated nose piece 36. The jaw shaft 38 terminates distally at a mounting block 42 securely fixed to the end of the jaw shaft 38. The mounting block 42 is slidingly received within the distal end 32 of the axial through bore 28 to permit adjustment of the jaw shaft extension relative to the handle 20. A set screw 44 fixes the mounting block 42 to lock the jaw shaft in axial fixed relationship to handle 20. The sleeve 40 terminates distally at an actuator block 46 securely fixed to the end of the sleeve 40. The actuator block is slidingly received within the proximal end 30 of the axial through bore 28 for translating the sleeve 40 relative to the shaft 38. A slide actuator 48 is mounted on the outside the body tube 22 and connected to the actuator block 46 by a screw 50 to form an actuator assembly. A slot 52 in the sidewall of the body tube 22 allows the actuator assembly to move axially relative to the body tube 22. The actuator assembly is movable between a distal, jaw open, position and a proximal, jaw closed position. A drive spring 54 biases the actuator block 46 toward the jaw closed position. The distal end of the slide actuator 48 includes an inwardly projecting locking tab 56 that engages a notch 58 to lock the actuator assembly in the jaw open position. A locking spring 60 biases the proximal end 62 of the slide actuator 48 outwardly such that the slide actuator 48 tends to pivot around the screw 50. Thus, the locking spring biases the locking tab 56 into engagement with the notch 58 when the slide actuator 48 is in the jaw open position. Pressing the proximal end 62 of the slide actuator 48 inwardly against the locking spring 60 pivots the locking tab 56 out of the notch 58 and releases the actuator assembly so that the actuator assembly can move proximally toward the jaw closed position.

The proximal end 64 of the jaw shaft 38 terminates in two or more jaws. In the illustrative forceps 12, the jaw shaft terminates into a “Y”-shaped pair of cantilevered jaw arms 66 with a jaw 68 formed at the end of each jaw arm 66. The jaw arms 66 are resilient to permit repeated elastic deformation of the jaw arms 66 between a jaw open position as shown in FIG. 3 and a jaw closed position as shown in FIG. 1. The jaw arms 66 may be made of any resilient biocompatible material. For example, the jaw arms may be made of stainless steel.

The jaws 68 each include a jaw body 70 with a jaw back 72 and a jaw face 74. The jaw faces 74 oppose one another and grip objects when the jaws 68 are biased toward the jaw closed position. The jaw back 72 includes a cutter 76 for forming an exit wound upon withdrawal of the forceps 12 from the eye. The cutter 76 includes an elongated thin blade projecting outwardly from the jaw back 72 and extending from the proximal end 79 of the jaw 68 along the jaw back 72. The cutter 76 includes a distally facing cutting portion 78 that initiates cutting upon withdrawal of the forceps 12. In the illustrative forceps, the distally facing portion 78 tapers from the full cutter height to the surface of the jaw arm. When the jaws 68 are spaced apart, such as when they are gripping an object, they angle away from one another proximally. Preferably, each cutter 76 extends along the blade back far enough to engage the instrument entry sclerotomy and begin cutting with minimal stretching or tearing of the sclerotomy before the cutters reach the sclerotomy. The cutter 76 may also be hinged within the jaw back such that the cutter 76 is exposed when an object is gripped. In this manner, the cutter 76 is hinged on the jaw back 72 such that the cutter 76 is in an extended cutting position when the jaw faces 74 are relatively nearer to one another, and the cutter 76 is in a retracted position within the jaw back 72 when the jaw faces 74 are relatively farther from one another. The illustrative cutter 76 also includes a proximally facing cutting portion 80 that can open an undersized sclerotomy upon insertion of the forceps 12 into the eye.

The illustrative jaw faces 74 include a soft silicone insert molded to the jaw bodies 70. The soft jaw face 74 deforms to grip an object. As the jaw face 74 deforms, it conforms to the shape of a portion of the gripped object and increases the contact area between the jaw face 74 and the object. The conforming jaw face 74 also surrounds and interdigitates with irregularities in the object's surface to produce a secure grip. The soft jaw face 74 provides a particularly effective grip on hard, slippery, and/or irregular objects. The illustrative jaw faces 74 also include teeth 82 molded integrally with the jaw face and projecting outwardly. The teeth 82 provide multiple discrete thinned regions of greater flexibility than a flat soft jaw face. Each tooth 82 is highly, locally, deformable to conform to a particular portion of an objects surface. These soft teeth 82 are able to conform to small irregularities in an objects surface. They are also able to locally deform and grip delicate objects and portions of objects. The illustrative jaws 68 are formed from stainless steel tubing cut in half with the convex outer surface forming the jaw backs 72 and receiving the cutters 76 and the jaw faces 74 being molded into the concave inner surfaces.

The retractable magnetic probe 100 includes a handle 102 including a distal body tube 104 having a proximal end 106, a distal end 108, and a longitudinal axis 110 extending therebetween. The distal body tube 104 includes an axial through bore 112. An end cap 114 threads onto the distal body tube 104 to close the axial through bore 112 distally. A proximal body tube 116 includes a proximal end 118 and a distal end 120. The proximal body tube 116 defines an axial spring chamber 122 opening proximally. A reduced diameter axial through bore 124 communicates from the spring chamber 122 through to the distal end 120 of the proximal body tube 116. The proximal body tube 116 includes a radially projecting flange 123. A spring retainer 126 includes an elongated body 128 having a proximal end, a distal end, and an axial through bore 134. The proximal end defines a radially projecting head 136.

The proximal body tube 116 is mounted in relative axially translating relationship to the distal body tube 104 with the spring retainer 126. A spring 138 is placed over the elongated body 128 of the spring retainer 126 in contact with the head 136. The spring retainer 126 is inserted into the spring chamber 122 of the proximal body tube 116 and the elongated body 128 is pressed through the axial through bore 124 at the distal end 120 of the proximal body tube 116 against spring pressure. The distal end of the spring retainer 126 is threaded into the proximal end 106 of the distal body tube 104. The proximal and distal body tubes 116, 104 may be axially translated relative to one another with the proximal body tube 116 sliding over the spring retainer body 128 and the spring 138 biasing the proximal and distal body tubes 116, 104 together. A cannulated nose piece 140 threads into the proximal body tube 116 to reduce the spring chamber 122 opening proximally.

A sleeve 142 is coaxially mounted to the nose piece 140 in fixed axial relationship. A magnet shaft 144 slides coaxially through the sleeve 142 and extends distally through the spring retainer 126 and the distal body tube 104 to a position near the distal end of the distal body tube. The magnet shaft 144 includes a magnet 146 mounted at its proximal end. The magnet shaft 144 is axially adjustable to change the magnet 146 extension relative to the handle 102. A pair of set screws 148 locks the magnet 146 position axially relative to the handle 102. A plurality of circumferentially arranged levers 150 surround the handle. The levers 150 each define a proximal end 156, a distal end 158, and a resilient hinge 160. The levers 150 are retained at each end by a ring 152, 154 abutting the flange 152 and end cap 114 respectively. The levers 150 define a gripping surface 162 (FIG. 1). Upon being pressed inwardly, the levers bend at the hinge 160 tending to straighten the levers 150 and cause their proximal and distal ends 156, 158 to move apart. This in turn forces the distal body tube 104 and proximal body tube 116 apart causing the magnet shaft 144 to translate distally relative to the sleeve 142 to retract the magnet 146 into the sleeve 142. Upon relaxing pressure on the levers 150, the spring 138 biases the distal body tube 104 back toward the proximal body tube 116 driving the magnet shaft 144 proximally relative to the sleeve 142 and redeploying the magnet 146.

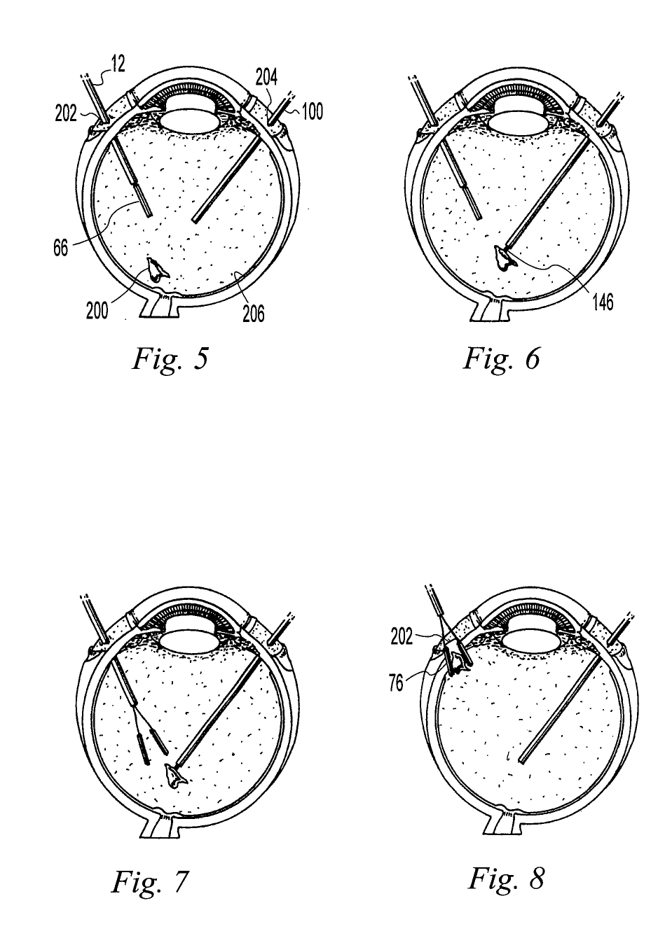

FIGS. 5-8 depict a surgical procedure for removing a foreign body 200 from the eye. The illustrative procedure depicts the forceps 12 and the retractable magnetic probe 100 being used together in a single procedure. This may be advantageous with a magnetic foreign body. However, the forceps 12 may be used alone for removing both magnetic and non-magnetic foreign bodies and the retractable magnetic probe 100 may be used alone for removing magnetic foreign bodies. For simplicity in describing the novel features of the invention, procedures commonly associated with ocular foreign body removal have been omitted such as imaging of the foreign body, immobilizing the globe of the eye, vitrectomy, use of a fluid tamponade, posterior vitreous detachment, peeling of membranes, intraocular lens implantation, suturing, laser treatment, and other procedures known in the art. Such other procedures may be employed in conjunction with the described novel procedure according to surgeon preference.

Referring to FIG. 5, a magnetic foreign body 200 has been traumatically deposited in the posterior segment of the eye. Sclerotomies 202, 204 are made on opposite sides of the eye to receive the forceps 12 and magnetic probe 100. The magnetic probe 100 is introduced into the eye with the magnet retracted by gently squeezing the gripping surface 162 of the levers 150. It is preferred to introduce the magnet in the retracted position to avoid unintentional attraction and movement of magnetic foreign bodies which could result in further trauma to the eye. Once the magnetic probe 100 is positioned adjacent the foreign body 200, the magnet 146 is extended by releasing pressure on the gripping surfaces 162. With the foreign body 200 engaged with the magnet 146, it is transferred to a more central position within the eye (FIG. 6). With the foreign body 200 moved safely away from the retinal surface 206, the forceps 202 are brought to the foreign body 200. The slide actuator 48 is moved distally to retract the sleeve 40 and allow the jaws 68 to spring open (FIG. 7). The slide actuator 48 may be locked in the open position by engaging the locking tab 56 in the locking notch 58. The jaws 68 are positioned around the foreign body 200 and the sliding actuator is moved proximally to extend the sleeve 40 and press the jaws 68 together to grip the foreign body 200. The soft jaw faces 74 deform to grip the foreign body 200 securely. The drive spring 54 maintains closing pressure on the jaws 68 absent finger pressure from the user. The forceps 12 and foreign body 200 are withdrawn through the entry sclerotomy 202 (FIG. 8). The view in FIG. 8 has been sectioned further around the forceps sclerotomy 202 to better illustrate the interaction of the forceps 12 and sclerotomy 202. As the jaws 68 reach the sclerotomy 202, the cutters 76 engage the edges of the sclerotomy and enlarge the sclerotomy to form an exit sclerotomy perfectly sized to pass the forceps jaws 68 and foreign body 200. No exit wound size estimation or separate exit wound cutting are required. Thus, multiple steps and opportunities for error are eliminated from the procedure.

Although examples of ocular foreign body removal instruments and their use have been described and illustrated in detail, it is to be understood that the same is intended by way of illustration and example only and is not to be taken by way of limitation. The invention has been illustrated as a sterile procedure pack including both a forceps and a retractable magnetic probe. However it is to be considered within the scope of this invention that either or both instruments could be produced as durable or multiple use instruments and/or provided separately. Likewise, the instruments have been illustrated as being constructed with particular male/female mating components, fixed and moving components, and with particular materials and fastening methods. However, in many instances it may be readily apparent that the male/female mating relationship may be reversed, the fixed and moving components may be reversed, and that alternative materials and fastening methods may be employed and the instruments still remain functional. Similarly, the instruments of this invention have been illustrated in use together to remove a magnetic foreign body. However, either instrument may be used alone to remove a magnetic foreign body and the forceps may be used alone to remove any kind of foreign body. Accordingly, these variations in and modifications to the ocular foreign body instruments and their use, and others, will be apparent to those of ordinary skill in the art, and the following claims are intended to cover all such modifications and equivalents.

What is claimed is:

1. An instrument set for performing ocular surgery, the set comprising:
a forceps having at least first and second jaws mounted for relative movement toward and away from one another, each jaw defining a jaw body having a jaw face directed generally toward the other jaw and a jaw back directed generally away from the other jaw, the jaws being movable between a first position in which the jaw backs are relatively nearer to one another and a second position in which the jaw backs are relatively further from one another, at least one of the jaws defining a cutter on its jaw back able to cut an exit wound upon withdrawal of the jaws from the eye corresponding in size to the spacing of the jaw backs in the second position.

2. The instrument set of claim 1 in which the forceps further comprise a handle, the jaws being mounted to the handle, the jaws being positioned proximally relative to the handle, the jaws each having a proximal end further from the handle and a distal end nearer to the handle, the cutter comprising an elongated blade projecting from the jaw back and extending along a length of the jaw back from the proximal end of the jaw back toward the distal end of the jaw back.

3. The instrument set of claim 1 in which the cutter is hinged on the jaw back such that the cutter is in an extended cutting position when the jaws are in a first position in which the jaw backs are relatively nearer to one another.

4. The instrument set of claim 2 in which the blade includes a distally directed cutting portion at its distal end able to initiate cutting upon withdrawal of the jaws from the eye.

5. The instrument set of claim 2 in which the blade includes a proximally directed cutting portion at its proximal end able to initiate cutting upon insertion of the jaws into the eye.

6. The instrument set of claim 1 in which at least one jaw face comprises a conforming portion comprising a material that is softer than the jaw body.

7. The instrument set of claim 6 in which the conforming portion comprises a polymer.

8. The instrument set of claim 7 in which the polymer comprises a polysiloxane.

9. The instrument of claim 6 in which the conforming portion is molded to the jaw body.

10. The instrument of claim 9 in which the jaw body comprises a member having a concave surface into which the conforming portion is molded and a convex surface defining the jaw back, the cutter projecting from the convex surface.

11. The instrument of claim 10 in which the jaw body comprises a semi-cylindrical member.

12. The instrument set of claim 6 in which the conforming portion comprises a conforming tooth projecting away from the jaw face.

13. The instrument set of claim 12 in which the conforming portion comprises a plurality of teeth projecting away from the jaw face, each tooth comprising a thin resilient body conformable to the shape of a portion of an object gripped between the jaws.

14. The instrument set of claim 1 further comprising:
an elongated probe comprising a sleeve having a first end and a second end, and a magnet translatable relative to the sleeve from a first position in which at least a portion of the magnet projects beyond the second end of the sleeve and a second position in which the magnet is retracted within the sleeve.

15. The instrument set of claim 4 in which the forceps and the probe are sterile and packaged together in a single procedure pack that encloses the forceps and probe in a closed sterile environment.

16. A method for removing an object from an eye, the method comprising:
providing a forceps having first and second jaws mounted for relative movement toward and away from one another, each jaw defining a jaw body having a jaw face directed generally toward the other jaw and a jaw back directed generally away from the other jaw, the jaws being movable between a first position in which the jaw backs are relatively nearer to one another and a second position in which the jaw backs are relatively further from one another;
introducing the jaws into the eye with the jaws in the first position;
grasping an object with the jaws in the second position; and withdrawing the forceps and object from the eye while simultaneously cutting an exit wound with a cutter mounted to the back of at least one of the jaws.

17. The method of claim 16 wherein providing a forceps comprises providing at least one jaw face with a conforming portion comprising a material that is softer than the jaw body and wherein grasping the object comprises pressing the conforming portion against the object to grip the object.

18. The method of claim 16 further comprising:
providing an elongated probe comprising a sleeve having a first end and a second end, and a magnet translatable relative to the sleeve from a first position in which at least a portion of the magnet projects beyond the second end of the sleeve and a second position in which the magnet is retracted within the sleeve;
introducing the second end of the sleeve into the eye;
attracting the object to the magnet; and
transferring the object from the magnet to the jaws of the forceps.

19. The method of claim 16 further comprising:
providing the forceps and probe packaged together in a single procedure pack that encloses the forceps and probe in a closed sterile environment.
